(19) United States
(12) Patent Application Publication
(10) Pub. No.: US 2026/0011339 A1
(43) Pub. Date: Jan. 8, 2026

(54) METHOD FOR PREDICTING DEPRESSION USING AI MODEL

(71) Applicant: Industry-Academic Cooperation Foundation, Chosun University, Gwangju (KR)

(72) Inventors: Jae Hyo JUNG, Gwangju (KR); Ji Sun HONG, Gwangju (KR); Ji Hun LEE, Gwangju (KR)

(73) Assignee: Industry-Academic Cooperation Foundation, Chosun University, Gwangju (KR)

(21) Appl. No.: 18/961,953

(22) Filed: Nov. 27, 2024

(30) Foreign Application Priority Data
Jul. 4, 2024 (KR) 10-2024-0088276

(51) Int. Cl. G10L 25/63; G10L 25/30

(52) U.S. Cl. CPC G10L 25/63; G10L 25/30

(57) ABSTRACT

The present disclosure relates to a method for determining whether there is depression through a voice signal of a user using an artificial intelligence (AI) model. A method for predicting depression according to an exemplary embodiment of the present disclosure includes: extracting, by a processor a mel-scale frequency cepstral coefficient (MFCC) of a training voice signal; training, by the processor, an autoencoder constituted by an encoder and a decoder using the extracted MFCC; training, by the processor, a classifier outputting a class according to there is the depression using a latent vector extracted by the encoder; and inputting, by the processor, a target MFCC extracted from a voice signal of a user into the autoencoder, and inputting a target latent vector extracted by the encoder into the classifier to evaluate whether there is the depression of the user.

### CROSS REFERENCE TO RELATED APPLICATION

The present application claims priority to Korean Patent Application No. 10-2024-0088276, filed Jul. 4, 2024, the entire contents of which are incorporated herein for all purposes by this reference.

### BACKGROUND

### Technical Field

The present disclosure relates to a method for determining whether there is depression through a voice signal of a user using an artificial intelligence (AI) model.

### Description of the Related Art

In the aftermath of the Corona 19 Pandemic, patients with mental diseases such as depression and stress are increasing. The depression is not only mental and physical pain, but also social isolation, and in serious cases, the depression can lead to suicide, so it is very important to diagnose or prevent the depression.

Existing methods of diagnosing depression are diagnosed by experts, such as psychiatrists and psychologists, through consultation on patients' symptoms, moods, and environments. However, the method has a problem that the method can make a wrong diagnosis because the method relies greatly on expert experience, level of questions, patient response accuracy and willingness to respond.

In recent years, an attempt to diagnose depression through analysis of electroencephalogram has been made to solve this problem, but the brainwave-based diagnostic method requires a high-performance sensor and a high-performance processor to analyze the sensor, so there is a limit that the general public cannot easily try at home without a doctor.

### SUMMARY

An object of the present disclosure is to evaluate whether there is depression using features inherent in a voice signal of a user.

The objects of the present disclosure are not limited to the above-mentioned objects, and other objects and advantages of the present disclosure that are not mentioned can be understood by the following description, and will be more clearly understood by embodiments of the present disclosure. Further, it will be readily appreciated that the objects and advantages of the present disclosure can be realized by means and combinations shown in the claims.

In order to achieve the object, a method for predicting depression using an AI model according to an exemplary embodiment of the present disclosure includes: extracting, by a processor a mel-scale frequency cepstral coefficient (MFCC) of a training voice signal; training, by the processor, an autoencoder constituted by an encoder and a decoder using the extracted MFCC; training, by the processor, a classifier outputting a class according to there is the depression using a latent vector extracted by the encoder; and inputting, by the processor, a target MFCC extracted from a voice signal of a user into the autoencoder, and inputting a target latent vector extracted by the encoder into the classifier to evaluate whether there is the depression of the user.

In an exemplary embodiment, the method further includes at least one of removing, by the processor, noise of the training voice signal, augmenting the training voice signal, and splitting the training voice signal according to a reference time interval.

In an exemplary embodiment, the training of the auto encoder includes performing unsupervised learning for the autoencoder using multi-dimensional data in which a size of the MFCC coefficient is defined over time.

In an exemplary embodiment, the autoencoder includes the autoencoder includes the encoder extracting the latent vector through at least one convolution layer, and a decoder reconstructing the MFCC from the latent vector through at least one deconvolution layer.

In an exemplary embodiment, the training of the classifier includes supervised learning the classifier by setting the latent vector to an input data of the classifier, and setting a class labeled on the training voice signal according to there is the depression to output data, and supervised learning the classifier.

In an exemplary embodiment, the artificial intelligence model is subject to end-to-end training so that an output of the encoder in the autoencoder is input into the classifier.

In an exemplary embodiment, the evaluating of there is the depression includes extracting the target MFCC from the voice signal of the user.

According to the present disclosure, an artificial intelligence model can be provided, which can evaluate whether there is depression using features inherent in a voice signal of a user, and as a result, there is an advantage in that the user can directly simply evaluate whether there is the depression without high accuracy without clinical and subjective judgment of an expert.

In addition to the above-described effects, the specific effects of the present disclosure are described together while describing specific matters for implementing the invention below.

### BRIEF DESCRIPTION OF THE DRAWINGS

FIG. 1 is a flowchart showing a method for predicting depression according to an exemplary embodiment of the present disclosure.

FIG. 2 is a flowchart showing a preprocessing process of a training voice signal according to an exemplary embodiment of the present disclosure.

FIG. 3 is a diagram illustrating a process of extracting MFCC from the training voice signal.

FIG. 4 is a diagram for describing a learning method of an autoencoder using MFCC.

FIG. 5 is a diagram illustrating a configuration of an autoencoder according to an exemplary embodiment of the present disclosure.

FIG. 6 is a diagram for describing a learning method of a classifier using a latent vector.

FIG. 7 is a diagram for describing a process of evaluating whether there is user's depression using an artificial intelligence model according to an exemplary embodiment of the present disclosure.

### DETAILED DESCRIPTION

The above-mentioned objects, features, and advantages will be described in detail with reference to the drawings, and as a result, those skilled in the art to which the present disclosure pertains may easily practice a technical idea of the present disclosure. In describing the present disclosure, a detailed description of related known technologies will be omitted if it is determined that they unnecessarily make the gist of the present disclosure unclear. Hereinafter, a preferable of the present disclosure will be described in detail with reference to the accompanying drawings. In the drawings, the same reference numeral is used for representing the same or similar components.

Although the terms “first”, “second”, and the like are used for describing various components in this specification, these components are not confined by these terms. The terms are used for distinguishing only one component from another component, and unless there is a particularly opposite statement, a first component may be a second component, of course.

Further, in this specification, any component is placed on the “top (or bottom)” of the component or the “top (or bottom)” of the component may mean that not only that any configuration is placed in contact with the top surface (or bottom) of the component, but also that another component may be interposed between the component and any component disposed on (or under) the component.

In addition, when it is disclosed that any component is “connected”, “coupled”, or “linked” to other components in this specification, it should be understood that the components may be directly connected or linked to each other, but another component may be “interposed” between the respective components, or the respective components may be “connected”, “coupled”, or “linked” through another component.

Further, a singular form used in the present disclosure may include a plural form if there is no clearly opposite meaning in the context. In the present disclosure, a term such as “comprising” or “including” should not be interpreted as necessarily including all various components or various steps disclosed in the present disclosure, and it should be interpreted that some component or some steps among them may not be included or additional components or steps may be further included.

In addition, in this specification, when the component is called “A and/or B”, the component means, A, B or A and B unless there is a particular opposite statement, and when the component is called “C or D”, this means that the term is C or more and D or less unless there is a particular opposite statement.

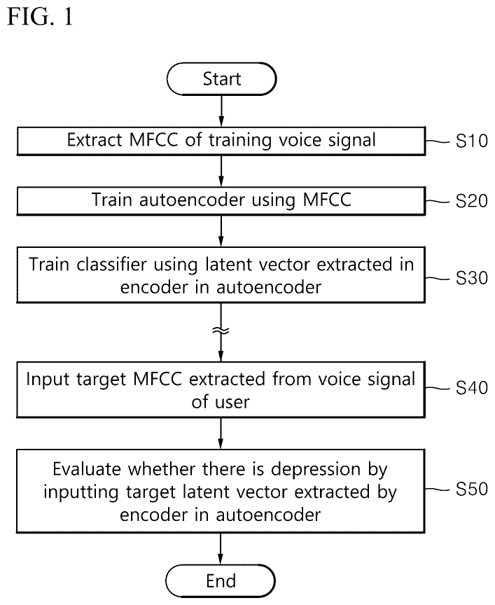

The present disclosure relates to a method for determining whether there is depression through a voice signal of a user using an artificial intelligence (AI) model. Hereinafter, a method for predicting depression according to an exemplary embodiment of the present disclosure will be described in detail with reference to FIGS. 1 to 7.

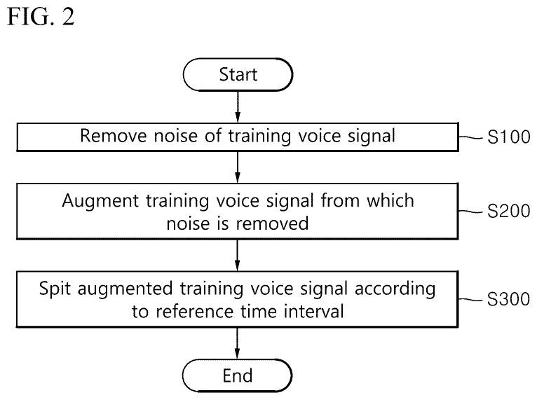

FIG. 1 is a flowchart showing a method for predicting depression according to an exemplary embodiment of the present disclosure. Further, FIG. 2 is a flowchart showing a preprocessing process of a training voice signal according to an exemplary embodiment of the present disclosure.

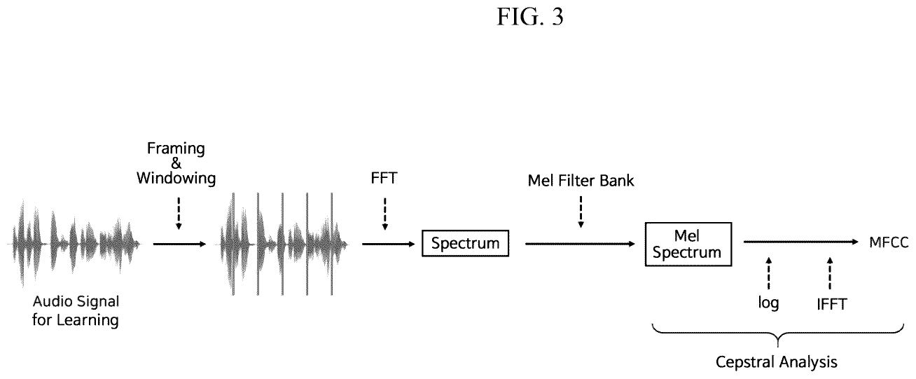

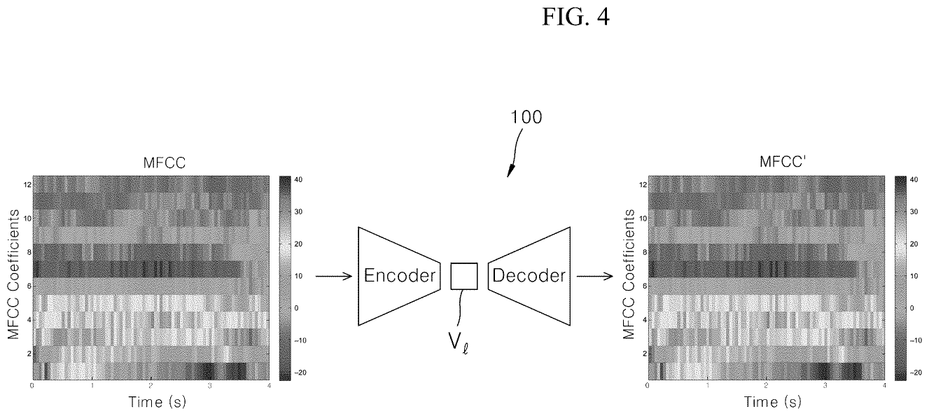

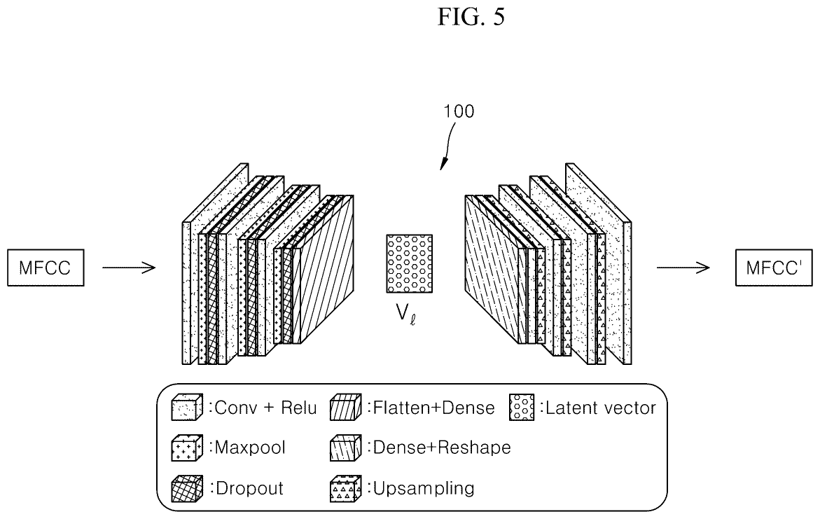

FIG. 3 is a diagram illustrating a process of extracting MFCC from the training voice signal, FIG. 4 is a diagram for describing a learning method of an autoencoder using MFCC, and FIG. 5 is a diagram illustrating a configuration of an autoencoder according to an exemplary embodiment of the present disclosure.

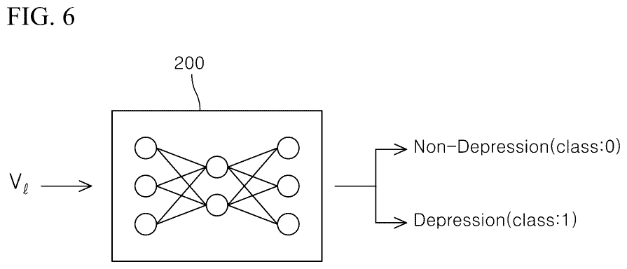

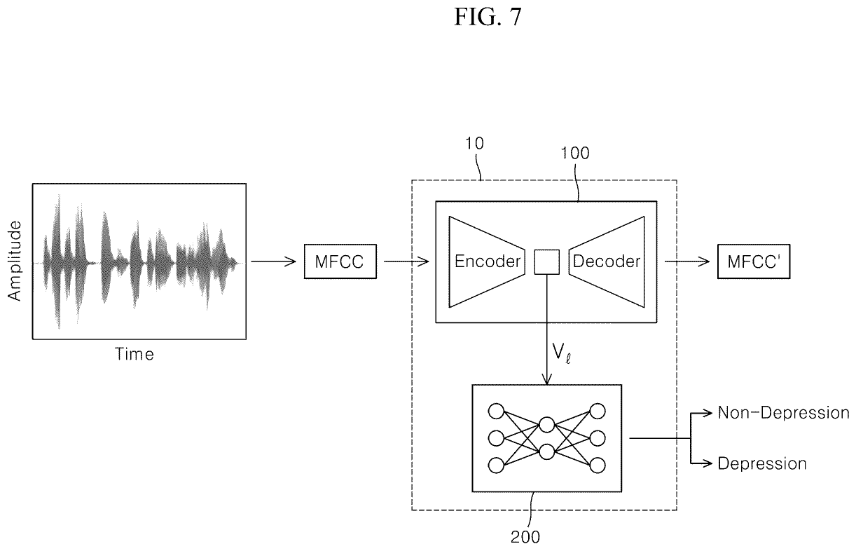

FIG. 6 is a diagram for describing a learning method of a classifier using a latent vector. Further, FIG. 7 is a diagram for describing a process of evaluating whether there is user's depression using an artificial intelligence model according to an exemplary embodiment of the present disclosure.

Referring to FIG. 1, the method for predicting depression according to an exemplary embodiment of the present disclosure relates to a method for predicting depression using an artificial intelligence model, and may be generally constituted by steps (S10 to S30) of training the artificial intelligence model, and a step (S40) of evaluating the depression using the artificial intelligence model performed after S10 to S30.

At this time, the step of training the artificial intelligence may include a step (S10) of extracting a mel-scale frequency cepstral coefficient (MFCC) of a training voice signal, a step (S20) of training an autoencoder using the MFCC, and a step (S30) of training a classifier using a latent vector extracted from an encoder in the autoencoder. Further, the depressing evaluating step may include a step (S40) of inputting a target MFCC extracted from a voice signal of a user into the autoencoder, and a step (S50) of evaluating whether there is depression by inputting a target latent vector extracted from the encoder in the autoencoder into the classifier.

However, the method for predicting depression illustrated in FIG. 1 follows an exemplary embodiment, and steps constituting the present disclosure are not limited to the exemplary embodiment illustrated in FIG. 1 and if necessary, some steps may be added, modified, or deleted.

The respective steps illustrated in FIG. 1 may be performed by a processor capable of computing and signal processing such as a central processing unit (CPU) and, a graphic processing unit (GPU), and to this end, the processor may further include at least one physical element among application specific integrated circuits (ASICs), digital signal processors (DSPs), digital signal processing devices (DSPDs), programmable logic devices (PLDs), field programmable gate arrays (FPGAs), a controller, and micro-controllers.

Hereinafter, the respective steps illustrated in FIG. 1 will be described in detail.

The processor may extract the MFCC of the training voice signal (S10). Here, the training voice signal may be a signal used for training the artificial intelligence model. The artificial intelligence model of the present disclosure may include the autoencoder and the classifier, and a learning method and a function of each element will be described below.

The training voice signal may be a signal indicating an intensity of sound over time, and may include voice signals of a patient who is diagnosed with depression, and a normal person. The training voice signal may be collected based on clinical data, and collected from a database built in advance in the technical field. In an example, the training voice signal may be collected from a distress analysis interview corpus (DAIC) data including a semi-structured interview collected by various methods.

Meanwhile, prior to extracting the MFCC from the training voice signal, the processor may preprocess the training voice signal. In the case of the above-described training voice signal, an interview performing time, an interview environment, etc., may vary depending on each signal, and standardization of the MFCC used for training may be required to enhance training efficiency of the artificial intelligence model, and to this end, the processor may apply the preprocessing to the training voice signal.

Referring to FIG. 2, the preprocessing process according to an exemplary embodiment of the present disclosure, may include a step (S100) of removing noise of the training voice signal, a step (S200) of augmenting the training voice signal from which the noise is removed, and a step (S300) of splitting the augmented training voice signal according to a reference time interval. In FIG. 2, it is illustrated that the respective steps are sequentially performed, but the respective steps are independently used in the preprocessing process, or two or more steps may be combined and performed.

Hereinafter, the respective steps illustrated in FIG. 2 will be described in detail.

The processor may remove the noise of the training voice signal (S100). The training voice signal collected as above may include not only a voice of an interviewer, but also noise generated from a surrounding environment. The noise distorts a sound quality in an MFCC extracting process to be described below, which may cause information on an actual voice to be lost, so the processor may remove the noise from the training voice signal.

In an example, the processor may remove the noise by a scheme of filtering a signal other than a frequency band corresponding to human vocalization, and to this end, may use a band pass filter (BPF).

Further, the processor may augment the training voice signal (S200). In general, the number of training voice signals generated from a normal person may be larger than the number of training voice signals generated from a depression patient. When all data are used for training the artificial intelligence model without adjusting the imbalance of data, the model may be biased-trained, and in this case, a prediction performance of the model may be deteriorated, so the processor may augment, particularly, the training voice signal generated from the depression patient.

In an example, the processor may generate an augmented signal by applying a time stretch technique which changes a speed or a length while maintaining a pitch of a voice as it is in order to augment the training voice signal. In another example, the processor may generate the augmented signal by applying a pitch shift technique which changes the pitch while maintaining the speed and the length of the voice as it is in order to augment the training voice signal.

Further, the processor may split the training voice signal according to the reference time interval (S300). Each training voice signal may have various lengths according to a response speed, breathing, silence, etc., of the interviewer. When the MFCC is extracted from voice signals having different lengths by the same scheme, a standard of the data (MFCC) used for training the artificial intelligence model is not unified, so a difficulty of training may be increased.

In order to prevent this, the processor may split the training voice signal according to the reference time interval, that is, a predetermined interval (e.g., 4 seconds). At this time, the processor may split the training voice signal so that a predetermined time (e.g., 0.5 seconds) is overlapped between the respective split voice signals to prevent information positioned at an edge of the voice signal from being lost.

Referring back to FIG. 1, the processor may extract the MFCC from the preprocessed training voice signal. Here, the MFCC may be a feature vector indicating a unique feature of sound in the voice signal, and may be obtained by applying cepstral analysis to mel spectrum.

When described with reference to FIG. 3, the processor may frame the preprocessed training voice signal into detailed intervals (e.g., 20 to 40 ms), and apply a window, e.g., a Hamming window in order to prevent frequency leakage of each framed detailed interval.

Subsequently, the processor applies Fourier transform, e.g., fast Fourier transform (FFT) to generate a spectrum for the training voice signal, and applies a mel filter bank to the spectrum to generate the mel spectrum.

Subsequently, the processor may generate the MFCC by a scheme of applying the cepstral analysis which applies inverse Fourier transform, e.g., inverse fast Fourier transform (IFFT), inverse discrete cosine transform (IDCT), etc., after taking a log in the mel spectrum.

Since the MFCC includes a unique feature (e.g., a speaking pattern, tone, voice energy, etc.) which the voice signal has in a temporal domain when human sound cognitive characteristics (mel spectrogram), the MFCC may be used as a first parameter for training the artificial intelligence model of the present disclosure.

Specifically, the processor may train an autoencoder 100 constituted by an encoder and a decoder by using the MFCC extracted through step S10 (S20).

The autoencoder 100 may perform an operation of extracting a feature by encoding input data, and reconstructing the input data by decoding the extracted feature. At this time, in order for the autoencoder 100 to extract a spatial feature and a pattern included in the voice signal, the processor extends a dimension of the MFCC extracted through step S10 to convert the MFCC into multi-dimensional data.

When FIG. 4 is described as an example, the processor may extend the dimension of the MFCC extracted in step S10, and convert the MFCC into multi-dimensional data in which a size for each MFCC coefficient is defined over time, and perform unsupervised learning for the autoencoder 100 by using the multi-dimensional data.

Specifically, the processor may input the multi-dimensional data into the autoencoder 100, and the autoencoder 100 may extract a latent vector vₗ from the corresponding multi-dimensional data through the encoder. Subsequently, the autoencoder 100 may reconstruct the multi-dimensional data from the latent vector vi through the decoder, and at this time, the processor may set a loss function which is in proportion to a difference between the data MFCC and the data MFCC′ reconstructed by the autoencoder 100, and as a result, the autoencoder 100 may be automatically trained so that the loss function becomes minimum, that is, the difference between the input data MFCC and the reconstructed data MFCC′.

Meanwhile, in order to extract the latent vector vi from the multi-dimensional data, and reconstruct the multi-dimensional data from the latent vector vi, the autoencoder 100 may include at least one convolution layer and a deconvolution layer.

When FIG. 5 is described as an example, the autoencoder 100 is a model based on a convolutional neural network (CNN), and an internal encoder may include a structure in which pairs constituted by a convolution layer, a pooling layer, and a dropout layer are sequentially connected. At this time, sizes of filters (or kernels) applied to respective in-pair convolution layers are sequentially increased, so the encoder may extract the latent vector vi including a spatial feature of the voice signal from the MFCC.

Further, the internal decoder of the autoencoder 100 may include a structure in which pairs constituted by the deconvolution layer and an upsampling layer are sequentially connected. At this time, sizes of filters (or kernels) applied to respective in-pair deconvolution layers are sequentially decreased, so the decoder may reconstruct the MFCC by gradually extending the latent vector vi.

Since a process in which the convolution based neural network extracts the latent vector vi and a process in which the deconvolution based neural network reconstructs the data from the latent vector vi follow the method known in the technical field, a detailed description will be omitted herein.

Through the above-described structure and training process, the encoder in the autoencoder 100 may extract the latent vector vi so that most important elements are included for data reconstruction, and the processor may train the classifier which outputs a class according to whether there is the depression by using the latent vector vi (S30). As described above, since the latent vector vi includes unique features (e.g., sound, intensity, tone, pitch, and a combination thereof) having the voice signal in a spatial domain, the latent vector vi may be used as a second parameter for training the artificial intelligence model of the present disclosure.

The classifier 200 may include any neural network that performs a classification task, and may be subject to supervised learning by the processor in order to perform the corresponding task. Since labeling for output data corresponding to the input data (latent vector vi) is required for the supervised learning, the processor may use a class labeled with the training voice signal collected in step S10 as output data of the classifier 200.

Whether there is the depression may be stored in the database collecting the training voice signal to correspond to each voice signal, and the processor may label each training voice signal with the class corresponding to whether there is the depression, e.g., a class of ‘0’ for non-depression and a class of ‘1’ for depression.

In a specific example, a personal Health Questionnaire Depression Scale (PHQ-8) score based on a response of the interviewer may be stored in the DAIC database jointly with the voice signal, and the processor may label a training voice signal having a PHQ-8 score of 0 to 9 points with 0 which is the class corresponding to the non-depression and a training voice signal having a PHQ-8 score of 10 points or more with 1 which is the class corresponding to the depression.

Subsequently, referring to FIG. 6, the processor sets, as the input data of the classifier 200, the latent vector vi extracted by the encoder, and sets, as the output data of the classifier 200, the labeled class (non-depression: 0 and depression: 1) to perform the supervised learning for the classifier 200.

According to the supervised learning, the classifier 200 may determine a correlation between the latent vector vi and whether there is the depression. For example, when the classifier 200 includes multi-layer perceptron (MLP), a parameter (weight) and a bias of each node constituting the MLP may be updated so that a prediction value of the classifier 200 is equal to the labeled class according to repetition of the training.

Meanwhile, the artificial intelligence model of the present disclosure, which includes the autoencoder 100 and the classifier 200 is configured to input an output of the encoder in the autoencoder 100 into the classifier 200 to be subject to end-to-end training.

When FIG. 7 is described as an example, the artificial intelligence model 10 according to the present disclosure may include the encoder 100 and the classifier 200, and may have a structure in which the output of the internal encoder of the autoencoder 100, that is, the latent vector vi is input into the classifier 200. At this time, since the autoencoder 100 is subject to unsupervised learning as described above, the entirety of the artificial intelligence model 10 may be subject to end-to-end training by a scheme of inputting the MFCC into the autoencoder 100 and setting the output of the classifier 200 to the labeled class.

When training the artificial intelligence model 10 is completed according to steps S10 to S30 described above, the processor may evaluate whether there is the user's depression using the corresponding model. Specifically, the processor may input a target MFCC extracted from the voice signal of the user into the autoencoder of which training is completed (S40), and inputs a target latent vector extracted by the encoder in the autoencoder 100 into the classifier 200 of which training is completed to evaluate whether there is the user's depression (S50).

The processor may first collect a voice signal of a user which becomes a depression evaluation target, and extract the target MFCC from the corresponding voice signal. Since the extracted target MFCC is an element input into the pre-trained autoencoder 100, the target MFCC may be extracted in the same scheme as the MFCC used for training the autoencoder 100, that is, the scheme as in step S10. As a result, it is natural that the preprocessing such as the noise removal (S100), the signal splitting (S300), etc., even in the voice signal of the user.

Subsequently, the processor may input the target MFCC into the autoencoder 100. The autoencoder 100 may extract the target latent vector from the target MFCC through the internal encoder, and the processor may input the target latent vector into the classifier 200.

As described in step S30, the classifier 200 learns the correlation between the latent vector and whether there is the depression by the supervised learning, so the classifier 200 may receive a target latent vector which is not used for the learning, and output a probability for whether there is the depression.

For example, as illustrated in FIG. 6, the trained classifier 200 may output a probability value corresponding to class 1 to be high when the feature included in the voice signal of the user is similar to the feature included in the training voice signal collected from the depression patient. For example, the classifier 200 may output a probability value corresponding to class 0 to be high when the feature included in the voice signal of the user is similar to the feature included in the training voice signal collected from the normal person.

The processor may evaluate whether there is the depression of the user based on the probability for each class output by the classifier 200. In a specific example, the processor may evaluate that the user has the depression when the probability value of the class corresponding to the depression is equal to or more than a reference value (e.g., 0.8). On the contrary, the processor may evaluate that the user is normal when the probability value of the class corresponding to the non-depression is less than the reference value (e.g., 0.4). Meanwhile, the processor my not evaluate the depression for a probability range (e.g., 0.4 or more or less than 0.8) in which evaluation is vague.

According to the present disclosure, the artificial intelligence model can be provided, which can evaluate whether there is the depression using features inherent in the voice signal of the user, and as a result, there is an advantage in that the user can directly simply evaluate whether there is the depression without high accuracy without clinical and subjective judgment of an expert.

Although the present disclosure has been described above by the drawings, but the present disclosure is not limited by the exemplary embodiments and drawings disclosed in the present disclosure, and various modifications can be made from the above description by those skilled in the art within the technical ideas of the present disclosure. Moreover, even though an action effect according to a configuration of the present disclosure is explicitly disclosed and described while describing the exemplary embodiments of the present disclosure described above, it is natural that an effect predictable by the corresponding configuration should also be conceded.

What is claimed is:

1. A method for predicting depression using an AI model, the method comprising:
extracting, by a processor a mel-scale frequency cepstral coefficient (MFCC) of a training voice signal;
training, by the processor, an autoencoder constituted by an encoder and a decoder using the extracted MFCC;
training, by the processor, a classifier outputting a class according to there is the depression using a latent vector extracted by the encoder; and
inputting, by the processor, a target MFCC extracted from a voice signal of a user into the autoencoder, and inputting a target latent vector extracted by the encoder into the classifier to evaluate whether there is the depression of the user.

2. The method for predicting depression of claim 1, further comprising:
at least one of removing, by the processor, noise of the training voice signal, augmenting the training voice signal, and splitting the training voice signal according to a reference time interval.

3. The method for predicting depression of claim 1, wherein the training of the autoencoder includes extending a dimension of the MFCC, and converting the MFCC into multi-dimensional data, and performing unsupervised learning for the autoencoder using the multi-dimensional data.

4. The method for predicting depression of claim 1, wherein the autoencoder includes the encoder extracting the latent vector through at least one convolution layer, and a decoder reconstructing the MFCC from the latent vector through at least one deconvolution layer.

5. The method for predicting depression of claim 1, wherein the training of the classifier includes supervised learning the classifier by setting the latent vector to an input data of the classifier, and setting a class labeled on the training voice signal according to there is the depression to output data, and supervised learning the classifier.

6. The method for predicting depression of claim 1, wherein the artificial intelligence model is subject to end-to-end training so that an output of the encoder in the autoencoder is input into the classifier.

7. The method for predicting depression of claim 1, wherein the evaluating of there is the depression includes extracting the target MFCC from the voice signal of the user.
